(19) United States
(12) Patent Application Publication
(10) Pub. No.: US 2018/0318436 A1
(43) Pub. Date: Nov. 8, 2018

(54) METHODS AND MEANS FOR INDUCING AN IMMUNE RESPONSE

(71) Applicant: BIONTECH AG, Mainz (DE)

(72) Inventor: Steve PASCOLO, Zürich (CH)

(21) Appl. No.: 15/763,601

(22) PCT Filed: Oct. 20, 2016
(86) PCT No.: PCT/EP2016/075156

§ 371 Date: Mar. 27, 2018

(30) Foreign Application Priority Data
Oct. 21, 2015 (EP) PCT/EP2015/074389

(51) Int. Cl. A61K 47/64; A61P 37/04; A61K 9/51; A61K 39/00; A61P 35/00

(52) U.S. Cl. CPC A61K 47/6455; A61P 37/04; A61K 9/513; A61K 2039/545; A61P 35/00; A61K 2039/55555; A61K 39/001141

(57) ABSTRACT

The present invention relates to a method for inducing an immune response manifested by type I interferon production in a synergistic manner comprising the sequential administration of danger signals within a certain timeframe and means for practicing the method. The present invention is particularly useful for immunomodulation, immunotherapy and vaccination.

The present invention relates to a method for inducing an immune response comprising two consecutive administrations of danger signals, in particular particles comprising Protamine and RNA, and means for performing the method. The method of the present invention is particularly useful for immunomodulation and vaccination.

### BACKGROUND OF THE INVENTION

Immune cells can be stimulated through triggering of receptors such as Toll Like Receptors or cytosolic receptors (e.g. RIG-I) by molecules or complexes termed “danger signals”. One of these danger signals is RNA. It can be recognized by TLR7, TLR8, TLR3 and TLR13 as well as by RIG-I and MDA-5. It was previously shown that under specific conditions (i.e. low salts) Protamine and RNA can generate nanoparticles which can deliver RNA in cells, stimulating immune receptors and thereby inducing activation of immune cells, i.e. maturation of cells and secretion of cytokines (Rettig L. et al., 2010, Blood 115, 4533-4541 and WO 2009/144230). These particles can activate TLR7 when taken up by for example plasmacytoid dendritic cells or TLR8 when taken up by for example monocytes.

The present invention is based on the observation that Protamine-RNA particles, particularly those bellow 450 nm, induce a moderate immune response when injected once intravenously. However, when they are injected twice within a time range of less than 6 hours they induce a strong immune response as mirrored for example by a high production of type I interferon, in particular interferon-alpha. Of note, inflammatory cytokines such as TNF-alpha are not synergistically induced by this injection protocol. We further observed that this synergistic induction of interferon-alpha depends on the interferon-α/β receptor (IFNAR). In addition, we demonstrated that the double injection protocol can cure established tumors in mice while bulk single injection of the total dose of Protamine-RNA particles does not. In addition, high interferon-alpha induction in vivo is seen when Protamine-RNA particles are injected 2 hours after another danger signal, for example double stranded RNA (dsRNA, ligand of TLR3), imiquimod (ligand of TLR7) or CpG DNA (ligand of TLR9). Thus, enhanced type I interferon production after Protamine-RNA particle injection is achieved by pre-treatment within less than 6 hours using injection of Protamine-RNA particles or other danger signals.

### SUMMARY OF THE INVENTION

In a first aspect, the present invention relates to a method for inducing an immune response in a subject comprising administering to said subject a second composition after administration of a first composition, the first composition and the second composition each comprising (i) a danger signal; and (ii) a pharmaceutically acceptable carrier, wherein the first composition and the second composition are administered within a time period such that a synergistic immune response is induced.

In one embodiment, the time period within which the first composition and the second composition are administered is about 7 hours, preferably about 6 hours, about 5 hours, about 4 hours, about 3 hours, about 2 hours, about 1 hour, or about 30 minutes. In one embodiment, the second composition is administered 7 hours or less, 6 hours or less, 5 hours or less, 4 hours or less, 3 hours or less, 2 hours or less, or 1 hour or less after administration of the first composition. In one embodiment, the second composition is administered at least 15 min, at least 30 min, at least 1 hour, or at least 1.5 hours after administration of the first composition. In one embodiment, the second composition is administered within a time period of 0.5 to 7 hours or 1 to 6 hours after administration of the first composition.

In one embodiment, the danger signal is a Toll-like receptor agonist and/or the danger signal induces type I interferon. In one embodiment, the Toll-like receptor agonist is selected from the group consisting of RNA including single-stranded RNA and double-stranded RNA; a particle comprising RNA, in which the RNA is associated with a cationic polymer or lipid or both a cationic polymer and lipid; unmethylated DNA containing CpG motifs; imiquimod; and resiquimod. In one embodiment, the cationic polymer is selected from the group consisting of Protamine, polyethyleneimine, poly-L-lysine, poly-L-arginine and histone. In one embodiment, the particle comprises RNA and Protamine. In one embodiment, the Protamine-RNA particle is a Protamine-RNA nanoparticle having a size in the range from about 10 nm to about 990 nm, from about 10 nm to about 750 nm, from about 10 nm to about 450 nm, from about 50 nm to about 450 nm, from about 50 nm to about 100 nm, or from about 90 nm to about 110 nm. In one embodiment, the Protamine-RNA nanoparticle has a Protamine:RNA mass ratio in the range from about 16:1 to about 1:2, from about 8:1 to about 1:2, or from about 4:1 to about 1:2.

The danger signal in the first and the second composition may be identical or different. In one embodiment of the method of the invention, the danger signal in the first and second composition is a Toll-like receptor (TLR) agonist. In one embodiment of the method of the invention, the danger signal in the first composition is a TLR3, 7, 8, 9 and/or 13 agonist and the danger signal in the second composition is a TLR7 and/or 8 agonist. In one embodiment of the method of the invention, the danger signal in the first and/or second composition is a Protamine-RNA nanoparticle.

In one embodiment, the RNA is an oligonucleotide or a messenger RNA. In one embodiment, the RNA comprises at least one U nucleotide or at least one G nucleotide, or at least one U nucleotide and at least one G nucleotide. In one embodiment, the RNA is modified RNA.

In one embodiment of the method of the invention, the induced immune response is detected by an at least 2-fold, at least 3-fold, at least 4-fold, at least 5-fold, at least 6-fold, at least 7-fold, at least 8-fold, at least 9-fold, or an at least 10-fold increase in type I interferon expression in serum obtained from the subject after administration of both the first composition and the second composition (sequential administration) as compared to administration of only one of the compositions and/or as compared to the simultaneous administration of both compositions, wherein the type I interferon preferably is interferon-alpha.

In one embodiment of the method of the invention, the induced immune response shows no increase or a decrease in TNF-alpha expression in serum obtained from the subject after administration of both the first composition and the second composition (sequential administration) as compared to the simultaneous administration of both compositions.

In one embodiment the second composition follows injection of recombinant or purified interferon

In one embodiment of the method of the invention, the first composition and/or the second composition further comprises an antigen.

In a further aspect, the present invention relates to a kit comprising a first container and a second container, wherein the first container contains a first composition comprising (i) a danger signal; and (ii) a pharmaceutically acceptable carrier; and the second container contains a second composition comprising (i) a danger signal; and (ii) a pharmaceutically acceptable carrier, and preferably the kit further comprises instructions that the first composition and the second composition are administered within a time period such that a synergistic immune response is induced.

In a further aspect, the present invention relates to a first composition and a second composition for use in inducing an immune response in a subject, wherein the second composition is administered after administration of the first composition, the first composition and the second composition each comprising (i) a danger signal; and (ii) a pharmaceutically acceptable carrier, wherein the first composition and the second composition are administered within a time period such that a synergistic immune response is induced.

In a further aspect, the present invention relates to a use of a first composition and a second composition for therapeutic use, wherein the second composition is administered after administration of the first composition, the first composition and the second composition each comprising (i) a danger signal; and (ii) a pharmaceutically acceptable carrier, wherein the first composition and the second composition are administered within a time period such that a synergistic immune response is induced. In one embodiment, the therapeutic use is treating cancer.

Embodiments of the kit of the invention, the first composition and the second composition for the use of the invention and the use of the invention are as described herein for the method of the invention.

The compositions described herein may be pharmaceutical compositions and may optionally comprise diluents, and/or excipients. The compositions may further comprise at least one adjuvant such as an oil and/or at least one antigen.

Following contacting of appropriate cells or administration to a subject, the danger signals described herein such as Protamine-RNA particles are capable of inducing interferon-alpha. Thus, the present invention is useful for stimulating the immune system, which stimulation of the immune system preferably involves the stimulation of one or more of TLR7, TLR8 and TLR3, preferably TLR7 and/or TLR8.

Administration of the first composition and/or the second composition may be intra-venous, sub-cutaneous, intra-muscular or intra-tumoral.

### BRIEF DESCRIPTION OF THE DRAWINGS

Other objects, advantages and novel features of the present invention will become apparent from the following detailed description when considered in conjunction with the accompanying drawings.

FIG. 1: A Double Injection of Protamine-RNA Particles induces Synergistic Production of Type I Interferon but not a TNF-alpha.

RNA (mRNA coding for firefly luciferase) and Protamine were diluted to 0.5 mg/ml using pure water. Hundred micrograms RNA (200 microliters) were mixed with hundred micrograms Protamine IPEX (200 microliters) thereby generating approximately 100 nm particles. Fourty percent Glucose was added to reach 5% glucose final and 5% Glucose was added so that the final injectable solution contains 10 micrograms RNA (and 10 micrograms Protamine) in 70 microliters. Mice received 70 microliters intravenous and 6 hours (“6 h delay”) or 5 hours (“5 h delay”) or 4 hours (“4 h delay”) or 2 hours (“2 h delay”) later, again 70 microliters of Protamine-RNA. Sera were drawn 3 hours after the second injection. As controls, mice received 140 microliters of the Protamine-RNA solution intravenous once (“Single Injection”). As negative control, mice received intravenous 100 microliters of Glucose 5% (“Glucose 5%). For control mice, sera were drawn three hours after injection. Three mice per group. Interferon-alpha was measured in sera using Verikine™ ELISA kit from PBL and TNF-alpha was measured using eBioscience ELISA kits. The results indicate that two injections of 10 micrograms of RNA in Protamine particles (10 micrograms of Protamine) are inducing several times more interferon-alpha in vivo than one injection of 20 micrograms of RNA in Protamine particles (20 micrograms of Protamine) particularly when the second injection is made 2 hours or 4 hours after the first one. More than 6 hours between injections do not give the synergistic effect on induction of interferon-alpha. There is no synergy when looking at TNF-alpha in serum: the single injection of 20 micrograms of RNA in Protamine particles (20 micrograms of Protamine) is actually more efficacious than two injections (2 hours or 4 hours or 5 hours or 6 hours apart) of 10 micrograms of RNA in Protamine particles (10 micrograms of Protamine) for induction of TNF-alpha.

FIG. 2: Synergy of the Double Injection for Induction of Type I Interferon Depends on Interferon (Alpha and Beta) Receptor 1 (IFNAR1)

RNA (mRNA coding for firefly luciferase) and Protamine were diluted to 0.5 mg/ml using pure water. Hundred micrograms RNA (200 microliters) were mixed with hundred micrograms Protamine IPEX (200 microliters) thereby generating approximately 100 nm particles. Fourty percent Glucose was added to reach 5% glucose final and 5% Glucose was added so that the final injectable solution contains 10 micrograms RNA (and 10 micrograms Protamine) in 70 microliters. Wild type (“BALB/c”, three animals) or IFNAR1 KO (“IFNAR KO”, two animals) mice received 70 microliters intravenous and after 2 hours again 70 microliters of Protamine-RNA intravenous. Sera were drawn 3 hours after the second injection. As negative control, we used serum from an untreated mouse “Uninjected”. Interferon-alpha was measured in sera using Verikine™ ELISA kit from PBL and TNF-alpha was measured using eBioscience ELISA kits. The results indicate that the synergistic induction of interferon-alpha in vivo by the two-injection-procedure requires IFNAR1: the synergy is not seen in IFNAR1 KO mice. Thus, a feed back loop through IFNAR1 allows the first Protamine-RNA particle injection to “sensitize” the immune system to respond strongly to a second injection.

FIG. 3: Imiquimod and CpG DNA can Sensitize to the Subsequent Injection of Protamine-RNA Particles

The DNA oligonucleotide “CpG 1585” (invivogen) which is a mouse TLR9 agonist was diluted at 0.5 micrograms per microlitre in PBS. Imiquimod (invivogen) was diluted to 0.1 microgram per microliter in PBS. RNA (mRNA coding for firefly luciferase) and Protamine were diluted to 0.5 mg/ml using pure water. Hundred micrograms RNA (200 microliters) were mixed with hundred micrograms Protamine IPEX (200 microliters) thereby generating approximately 100 nm particles. Fourty percent Glucose was added to reach 5% glucose final and 5% Glucose was added so that the final injectable solution contains 10 micrograms RNA (and 10 micrograms Protamine) in 70 microliters.

Mice were injected intravenous with 100 microliters of CpG (50 micrograms DNA “CpG(2h)PR11”) or 100 microliters of imiquimod (10 micrograms Imiquimod “Imi(2h)PR11”) or 70 microliters of Protamine-RNA particles (10 micrograms RNA “PR11(2h)PR11”) and 2 hours later all received 70 microliters of Protamine-RNA (10 micrograms RNA) intravenous. Three mice per group. Sera were drawn 3 hours after this second injection. As controls, mice were injected intravenous with 100 microliters of CpG (50 micrograms “CpG alone”) or 100 microliters of imiquimod (10 micrograms “Imi alone”) or 70 microliters of Protamine-RNA particles (10 micrograms RNA “PR11 alone”). Three mice per group. Sera were drawn 3 hours after the injection. Interferon-alpha was measured in sera using Verikine™ ELISA kit from PBL. The results indicate that the injection of a TLR9 agonist (CpG DNA) or TLR7 agonist (Imiquimod) can sensitize the immune system to produce an enhanced amount of interferon-alpha after injection of Protamine-RNA particles when compared to a single injection of Protamine-RNA alone.

FIG. 4: Double Stranded RNA (dsRNA) can Sensitize to the Subsequent Injection of Protamine-RNA

Double stranded RNA (dsRNA, invivogen) which is a mouse TLR3 agonist was diluted at 0.02 micrograms per microliter in PBS. Single stranded RNA (mRNA coding for firefly luciferase) and Protamine were diluted to 0.5 mg/ml using pure water. Hundred micrograms mRNA (200 microliters) were mixed with hundred micrograms Protamine IPEX (200 microliters) thereby generating approximately 100 nm particles. Fourty percent Glucose was added to reach 5% glucose final and 5% Glucose was added so that the final injectable solution contains 10 micrograms RNA (and 10 micrograms Protamine) in 70 microliters. Mice were injected intravenous with 100 microliters of dsRNA (2 micrograms dsRNA “dsRNA(2h)PR11”) or 70 microliters of Protamine-RNA (10 micrograms RNA “PR11(2h)PR11”) and 2 hours later all received 70 microliters of Protamine-RNA (10 micrograms RNA) intravenous. Three mice per group. Sera were drawn 3 hours after the second injection. As controls, mice were injected intravenous with 100 microliters of dsRNA (2 micrograms “dsRNA alone”) or 70 microliters of Protamine-RNA (10 micrograms RNA “PR11 alone”) and sera were drawn respectively 5 hours or 3 hours after the injection. Interferon-alpha was measured in sera using Verikine™ ELISA kit from PBL. The results indicate that the injection of a TLR3 agonist (dsRNA) can sensitize the immune system to produce an enhanced amount of interferon-alpha after injection of Protamine-RNA particles when compared to a single injection of Protamine-RNA alone.

FIG. 5: A Double Injection Schedule of Protamine-RNA Particles is Necessary to Cure Established Tumors

Single stranded RNA (mRNA coding for firefly luciferase) and Protamine were diluted to 0.5 mg/ml using pure water. Five hundred micrograms mRNA (1000 microliters) were mixed with five hundred micrograms Protamine IPEX (1000 microliters) thereby generating approximately 100 nm particles. Fourty percent Glucose was added to reach 5% glucose final and 5% Glucose was added so that the final injectable solution contains 10 micrograms RNA (and 10 micrograms Protamine) in 70 microliters.

On day 0, mice received intravenous 1 million of CT26/luciferase cells (mouse colon tumor cell line expressing firefly luciferase). At day 3 and day 10, mice were injected intravenous with 140 microliters of Protamine-RNA particles (20 micrograms RNA “One injection”) or 70 microliters of Protamine-RNA (10 micrograms RNA “PR11(2h)PR11”) twice within 2 hours (“Two injections”). A group of mice received at day 3 and 10, 100 microliters of 5% Glucose. Over time, the luciferase signal emitted by the mice (by tumor cells growing in lungs) was quantified using an in vivo imaging system. Ten mice per group. The results demonstrate that the double injection schedule (two injections within less than 6 hours) is required to stimulate anti-cancer immunity and obtain inhibition of tumor growth.

### DETAILED DESCRIPTION OF THE INVENTION

In the following, definitions will be provided which apply to all aspects of the present invention.

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., (1995) Helvetica Chimica Acta, CH-4010 Basel, Switzerland.

The practice of the present invention will employ, unless otherwise indicated, conventional methods of biochemistry, cell biology, immunology, and recombinant DNA techniques which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Particles form spontaneously when combining Protamine and RNA and the average size of those particles can range from about 50 nm to more than 1000 nm depending on reagent concentration and salt concentration in solutions used to formulate Protamine and RNA before mixing them. These particles stimulate Toll Like Receptors, such as TLR7 and/or TLR8 and thereby induce immune activation that can be monitored for example through measurement of production of cytokines such as interferon-alpha or TNF-alpha. The present inventor surprisingly observed that when injected intravenously into animals, the induction of interferon-alpha detectable in serum is moderate unless the particles are injected twice within a certain time period such as less than 6 hours. This enhanced interferon-alpha production is termed “synergistic” in the following. A synergistic effect is not detected when looking at other cytokines such as TNF-alpha: as much TNF-alpha is induced by a single or a double (within 6 hours) injection of Protamine-RNA particles.

As an alternative, danger signals, in particular TLR agonists, other than Protamine-RNA particles can be used instead of or in conjunction with Protamine-RNA particles according to the invention to get the synergistic effect. For example, an injection of a TLR3 (dsRNA) or TLR9 (CpG DNA) or TLR7 (imiquimode) ligand potentiates the interferon-alpha production induced by a subsequent (performed two hours later) injection of Protamine-RNA nanoparticles. it could also be postulated that instead of injection of danger signals, recombinant or purified interferons could be injected prior to Protamine-RNA particles to get the synergistic effect.

As the multiple injections induce a synergistic interferon-alpha production, it is expected to induce also a synergistic adaptive immunity against antigens that may be associated (added or linked) to the compositions described herein, in particular to the Protamine-RNA particles.

The term “immune response” refers to a response within an organism that preferably protects against disease. An immune response may be prophylactic and/or therapeutic. According to the invention an “induced immune response” may be reflected by an increased level of interferon-alpha.

“Inducing an immune response” may mean that there was no immune response before inducing an immune response, but it may also mean that there was a certain level of immune response before inducing an immune response and after inducing an immune response said immune response is enhanced. Thus, “inducing an immune response” includes “enhancing an immune response”. Preferably, after inducing an immune response in a subject, said subject is protected from developing a disease such as a cancer disease or the disease condition is ameliorated by inducing an immune response.

According to the invention, the term “synergistic immune response” in reference to the sequential administration of two compositions refers to the fact that the immune response resulting from said sequential administration of the two compositions is stronger as compared to the administration of only one of the compositions and/or as compared to the simultaneous administration of both compositions.

The term “danger signal” according to the invention generally relates to any substance or event that is able to activate immune cells such as dendritic cells (DCs) and therefore initiate or induce immune responses, including innate and adaptive immune responses. More specifically, the term relates to molecules, which are released during infections and/or tissue damage and cellular stress. The term “danger signal” includes molecular species either associated with pathogens (pathogen-associated molecular patterns; PAMPs) or directly derived from tissue injury damage-associated molecular patterns (damage-associated molecular patterns; DAMPs), or secreted by activated immune cells as amplifiers of the immune activation, inorganic materials and man-made technologies (e.g., nanomaterials) having the potential to activate immune cells directly or indirectly by inducing tissue damage and release of DAMPs and perturbations in tissue steady state including but not limited to hypoxia, changes in acidity, or osmolarity, and metabolic stress.

The term “pathogen-associated molecular pattern” or “PAMP” relates to molecular structures common to bacteria, viruses, or other microorganism, like lipopolysaccharide (LPS), flagellin, peptidoglycan and nucleic acids normally associated with viruses, such as double-stranded RNA (dsRNA), or unmethylated CpG motifs, that are able to activate immune cells such as DCs. These molecules are recognized by pattern recognition receptors (PRRs) such as Toll-like receptors (TLRs) expressed on both immune and non-immune cells.

The term “damage-associated molecular pattern” or “DAMP” relates to endogenous molecular structures that are normally contained within the cell interior and hidden from the immune system, and are liberated upon tissue damage. Examples include ATP, HSPs, and HMGB1. These molecules are recognized by a number of receptors, including PRRs, and are capable of inducing inflammation and immune responses in the absence of infection.

Danger signals include exogenous danger signals such as PAMPS and endogenous danger signals. Endogenous danger signals include primary endogenous danger signals and secondary endogenous danger signals.

Primary endogenous danger signals are endogenous molecules that are normally contained within the cell interior or present in an inactive form, hidden from the immune system, and mostly performing non-immune functions. They are released upon tissue damage and are able to activate immune cells such as DCs by triggering a number of receptors, including PRRs. Examples include nucleic acids, ATP, ADP, adenosine, uric acid, heat shock proteins (HSPs), high mobility group box protein 1 (HMGB1), Type I interferons (Type I IFNs), degradation products of the extracellular matrix (ECM), mitochondrial DNA, N-formyl peptides, acidity, osmolarity and hypoxia.

Secondary endogenous danger signals are endogenous molecules that are actively secreted by immune cells upon activation to mediate innate and adaptive immune activation in an autocrine and paracrine manner. Secondary endogenous danger signals include lymphocyte-derived activators of DCs such as CD40-L and granulysin, neutrophil-derived alarmins and pro-inflammatory cytokines such as TNF-α, Type I IFNs, and HMGB1.

In one embodiment, the term “danger signal” according to the invention relates to nucleic acids that are recognized by the immune system as danger signals, including DNA containing unmethylated CG sequences (CpG), unmodified single-stranded RNA (ssRNA), and double-stranded RNA (dsRNA). Such nucleic acids mislocalized in the intracellular compartments are preferably recognized by endosome-resident Toll-like receptors (TLRs) such as TLR9, TLR7, TLR8, and TLR3, respectively, which when engaged, deliver an activation signal to the cell. In one preferred embodiment, the term “danger signal” according to the invention relates to ssRNA stimulating TLR7 and TLR8, in particular ssRNA containing U-residues and/or G-residues. In one particularly preferred embodiment, the term “danger signal” according to the invention relates to such ssRNA formulated in Protamine particles.

If according to the invention a danger signal is a peptide or protein, such danger signal may be delivered to a subject as nucleic acid such as RNA encoding said peptide or protein, optionally present in particles as described herein, for expression of the nucleic acid within cells of the subject.

The Protamine-RNA particles described herein are preferably of less than 450 nm since those are most efficacious to induce interferon-alpha as previously demonstrated in vitro (Rettig et al, 2010). They can be formed for example by diluting Protamine and RNA in solutions containing less than 50 mM electrolytes and optionally further diluting using sugar-containing solutions in order to be in isotonic injectable form. Protamine-RNA particles may be further modified for example by pegylation. Production of interferon-alpha induced by Protamine-RNA particles probably relies on triggering of mostly TLR7 which is noticeably expressed in plasmacytoid dendritic cells, one of the cell type most potent in producing interferon-alpha. For generating immunostimulating particles the RNA molecules are preferably at least 10 residues in length and contain U-residues. The mass ratio of Protamine to RNA is preferably at least 0.5 (preferably not more than twice more RNA than Protamine). In preferred embodiments this ratio is 1 or higher (most preferred the same mass amount of Protamine and total RNA or more Protamine than total RNA is used), 2 or higher, 4 or higher and preferably up to 16, more preferably up to 8.

A preferred procedure for the preparation of particles of the invention containing Protamine as the cationic agent (or cationic polymer) comprises the steps of diluting Protamine and RNA at concentrations of less than approximately 5 mg/ml, at best at 1 mg/ml or less using pure water or low salt solution (preferably less than 50 mM electrolytes), mixing the two solutions and then optionally adding sugar containing solution so that the final formulation contains 5% sugar i.e. an osmolarity of approximately 300 mOsm/L.

Another preferred procedure for the preparation of particles of the invention containing Protamine as the cationic agent comprises the steps of diluting Protamine and RNA at concentrations of less than approximately 5 mg/ml, at best at 1 mg/ml or less using salt-free or low salt solutions (preferably less than 50 mM electrolytes) of 5% sugar and mixing the two solutions (the final formulation contains 5% sugar i.e. an osmolarity of approximately 300 mOsm/L).

In the context of the present invention, the term “RNA” relates to a molecule which comprises ribonucleotide residues and preferably is entirely or substantially composed of ribonucleotide residues. “Ribonucleotide” relates to a nucleotide with a hydroxyl group at the 2′-position of a β-D-ribofuranosyl group. The term “RNA” comprises isolated RNA such as partially or completely purified RNA, essentially pure RNA, synthetic RNA, and recombinantly generated RNA and includes modified RNA which differs from naturally occurring RNA by addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

RNA can be isolated from cells, can be made from a DNA template, or can be chemically synthesized using methods known in the art. In preferred embodiments, RNA is synthesized in vitro from a DNA template. In one particularly preferred embodiment, RNA, in particular mRNA, is generated by in vitro transcription from a DNA template. The in vitro transcription methodology is known to the skilled person. For example, there is a variety of in vitro transcription kits commercially available. In one particularly preferred embodiment, RNA is in vitro transcribed RNA (IVT RNA).

According to the invention, “RNA” includes mRNA, tRNA, rRNA, snRNAs, ssRNA and dsRNA.

According to the invention preferred as RNA are synthetic oligonucleotides of 6 to 100, preferably 10 to 50, in particular 15 to 30 or 15 to 20 nucleotides or messenger RNA (mRNA) of more than 50 nucleotides, preferably of 50 to 10,000, preferably 100 to 5000, in particular 200 to 3000 nucleotides.

According to the present invention, the term “mRNA” means “messenger-RNA” and relates to a “transcript” which may be generated by using a DNA template and may encode a peptide or protein. Typically, an mRNA comprises a 5′-UTR, a protein coding region, and a 3′-UTR. In the context of the present invention, mRNA may be generated by in vitro transcription from a DNA template.

According to the invention, “ssRNA” means single-stranded RNA and includes mRNA, tRNA, rRNA, snRNAs, and other ssRNAs. ssRNA may contain self-complementary sequences that allow parts of the RNA to fold and pair with itself to form double helices.

According to the invention, “dsRNA” means double-stranded RNA and is RNA with two partially or completely complementary strands. The size of the strands may vary from 6 nucleotides to 10000, preferably 10 to 8000, in particular 200 to 5000, 200 to 2000 or 200 to 1000 nucleotides.

There is no specific ribonucleotide sequence requirement for the RNA molecules to be suitable according to the present invention. However, it is not excluded that certain RNA sequences would provide best biological activities.

According to the invention, the stability of RNA may be modified as required. For example, RNA may be stabilized by one or more modifications having stabilizing effects on RNA.

The term “modification” in the context of RNA as used according to the present invention includes any modification of RNA which is not naturally present in said RNA.

In one embodiment of the invention, the RNA used according to the invention does not have uncapped 5′-triphosphates. Removal of such uncapped 5′-triphosphates can be achieved by treating RNA with a phosphatase.

In one embodiment, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap (m⁷G). In the context of the present invention, the term “5′-cap” includes a 5′-cap analog that resembles the RNA cap structure and is modified to possess the ability to stabilize RNA if attached thereto, preferably in vivo and/or in a cell.

Providing an RNA with a 5′-cap or 5′-cap analog may be achieved by in vitro transcription of a DNA template in the presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

The RNA may comprise further modifications. For example, a further modification of the RNA used in the present invention may be an extension or truncation of the naturally occurring poly(A) tail.

The term “stability” of RNA relates to the “half-life” of RNA. “Half-life” relates to the period of time which is needed to eliminate half of the activity, amount, or number of molecules. In the context of the present invention, the half-life of an RNA is indicative for the stability of said RNA.

Of course, if according to the present invention it is desired to decrease stability of RNA, it is possible to modify RNA so as to interfere with the function of elements as described above increasing the stability of RNA.

In one embodiment, the RNA described herein is RNA, in particular mRNA, encoding a peptide or protein. According to the invention, the term “RNA encoding a peptide or protein” means that the RNA, if present in the appropriate environment, preferably within a cell, can direct the assembly of amino acids to produce, i.e. express, the peptide or protein during the process of translation. Preferably, RNA according to the invention is able to interact with the cellular translation machinery allowing translation of the peptide or protein.

The term “expression” is used according to the invention in its most general meaning and comprises the production of RNA and/or peptides or proteins, e.g. by transcription and/or translation. With respect to RNA, the term “expression” or “translation” relates in particular to the production of peptides or proteins. It also comprises partial expression of nucleic acids. Moreover, expression can be transient or stable.

In the context of the present invention, the term “transcription” relates to a process, wherein the genetic code in a DNA sequence is transcribed into RNA. Subsequently, the RNA may be translated into protein. According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”.

The term “translation” according to the invention relates to the process in the ribosomes of a cell by which a strand of messenger RNA directs the assembly of a sequence of amino acids to make a peptide or protein.

Substances or vehicles with which RNA can be associated, e.g. by forming complexes with the RNA or forming vesicles in which the RNA is enclosed or encapsulated, preferably resulting in increased stability of the RNA compared to naked RNA, are contemplated for use with RNA described herein.

The carriers useful according to the invention include lipid-containing carriers such as cationic lipids, liposomes and micelles, cationic polymers such as DEAE dextran or polyethyleneimine and nanoparticles.

Cationic lipids may form complexes with negatively charged nucleic acids. Any cationic lipid may be used according to the invention. Cationic lipids and/or cationic polymers can be used to complex nucleic acids, thereby forming so-called lipoplexes, polyplexes and/or poly!ipop!exes, respectively, and these complexes have been shown to deliver nucleic acids into cells.

Liposomes are microscopic lipidic vesicles often having one or more bilayers of a vesicie-forming lipid, such as a phospholipid, and are capable of encapsulating a drug. Different types of liposomes may be employed in the context of the present invention, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bilayered forms known in the art. The size and lamellarity of the liposome will depend on the manner of preparation and the selection of the type of vesicles to be used will depend on the preferred mode of administration. Preferred injectable liposomes are those in the size range of 10-500, 20-400, 50-200, 50-150, 50-120, 50-100, or 50-90 nm in diameter. Cationic liposomes are structures that are made of positively charged lipids and are increasingly being used in gene therapy due to their favourable interactions with negatively charged nucleic acids and cell membranes. Cationic liposomes are also known as cationic lipoplexes. Liposomes should not be confused with micelles and reverse micelles composed of monolayers. The lipid assembly may be combined with stabilizers. Non-limiting examples of stabilizers include cholesterol and similar membrane active sterols, lipopolymers such as PEGylated lipids.

Formation of liposomes is not a spontaneous process. Lipid vesicles are formed when phospholipids such as lecithin are placed in water and consequently form one bilayer or a series of bilayers, each separated by water molecules, once enough energy is supplied. Liposomes may be formed using standard methods such as the reverse evaporation method (REV), the dehydration-rehydration method (DRV), sonication or other suitable methods. Liposomes can be created, for example, by sonicating phospholipids in water. Low shear rates create multilamellar liposomes, which have many layers. Continued high-shear sonication tends to form smaller unilamellar liposomes. In this technique, the liposome contents are the same as the contents of the aqueous phase. Sonication is generally considered a “gross” method of preparation as it can damage the structure of the drug to be encapsulated. Newer methods such as extrusion and Mozafari method are employed to produce materials for human use.

After liposome formation, the liposomes can be sized to obtain a population of liposomes having a substantially homogeneous size range, typically between about 10 and 500 nm.

According to the invention, Protamine is preferred as cationic carrier agent. The term “Protamine” refers to any of various strongly basic proteins of relatively low molecular weight that are rich in arginine and are found associated especially with DNA in place of somatic histones in the sperm cells of various animals (as fish). In particular, the term “Protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the invention, the term “Protamine” as used herein is meant to comprise any Protamine amino acid sequence obtained or derived from native or biological sources including fragments thereof and multimeric forms of said amino acid sequence or fragment thereof. Furthermore, the term encompasses (synthesized) polypeptides which are artificial and specifically designed for specific purposes and cannot be isolated from native or biological sources.

The Protamine used according to the present invention can be sulfated Protamine or hydrochloride Protamine. In a preferred embodiment, the Protamine source used for the production of the particles of the invention is Protamine 5000 which contains Protamine at more than 10 mg/ml (5000 heparin-neutralizing units per ml) in an isotonic salt solution and which is diluted as set forth above.

The particles preferably have a Protamine:RNA weight ratio from 16:1 to 1:2, preferably from 8:1 to 1:2, more preferably from 4:1 to 1:2.

The average “size” of the particles described herein is generally the “design size” or intended size of the particles prepared according to an established process. Size may be a directly measured dimension, such as average or maximum diameter, or may be determined by an indirect assay such as a filtration screening assay. Direct measurement of particle size is typically carried out by dynamic light scattering (also termed light scattering spectroscopy). As minor variations in size arise during the manufacturing process, a variation up to 40% of the stated measurement is acceptable and considered to be within the stated size i.e. 50 nm to 990 nm or preferably 50 nm to 450 nm in average. Alternatively, microcarrier size may be determined by filtration screening assays. For example, a particle preparation is less than a stated size, if at least 97% of the particles pass through a “screen-type” filter of the stated size.

Coating the Protamine-RNA particles with polyethyleneglycol (PEG) is one method that could help enhancing the bioavailability and thus the bioactivities of nanoparticles.

In accordance with one embodiment of the invention the particles described herein such as Protamine-RNA particles comprise on their outer surface a targeting agent or ligand such as an antibody which can selectively or preferably deliver the particles to a target cell population, and/or to a target organ or tissue. For example, liposomes bearing ligands can target receptors expressed on diseased cells. This ligand-binding promotes efficient drug uptake into cells and enhances efficacy. One targeting means which has been explored employs antibodies attached covalently or through electrostatic interactions to particle surfaces.

The ligand may be capable of binding to a disease-associated antigen such that the particles when administered accumulate at a diseased organ or tissue characterized by cells expressing the disease-associated antigen and preferably being characterized by association of the disease-associated antigen with their cell surface, e.g. the disease-associated antigen is a transmembrane protein. The disease-associated antigen may be a tumor-associated antigen and is preferably associated with the surface of a diseased cell such as a tumor cell but preferably not with the surface of a healthy cell, Preferably the ligand for site specific targeting binds to an extracellular portion of the disease-associated antigen.

In accordance with one embodiment of the invention the particles described herein such as Protamine-RNA particles are coated with an antigen (e.g. a peptide, a protein or a sugar) against which an adaptive immune response would be triggered.

In accordance with one embodiment, the invention envisions the use of endosome destabilising agents (EDA) that could favour delivery of particles described herein such as Protamine-RNA particles or of any other component included in the injected formulation (for example free mRNA) to the cytosole. The EDA can be for example a pH-reactive agent (polymers and peptides that may, for example, change their structural conformation upon exposure to a particular pH or pH range), a photosensitizer (the endosome destabilizing activity of the photosensitizer is triggered by exposure to light) or an external stimulus such as ultrasound.

The term “peptide” according to the invention comprises oligo- and polypeptides and refers to substances comprising two or more, preferably 3 or more, preferably 4 or more, preferably 6 or more, preferably 8 or more, preferably 10 or more, preferably 13 or more, preferably 16 more, preferably 21 or more and up to preferably 8, 10, 20, 30, 40 or 50, in particular 100 amino acids joined covalently by peptide bonds. The term “protein” preferentially refers to large peptides, preferably to peptides with more than 100 amino acid residues, but in general the terms “peptide” and “protein” are synonyms and are used interchangeably herein.

According to the present invention, RNA may encode a peptide or protein. Accordingly, RNA may contain a coding region (open reading frame (ORF)) encoding a peptide or protein. For example, RNA may encode and express an antigen or a pharmaceutically active peptide or protein such as an immunologically active compound (which preferably is not an antigen). In this respect, an “open reading frame” or “ORF” is a continuous stretch of codons beginning with a start codon and ending with a stop codon.

The term “pharmaceutically active peptide or protein” includes a peptide or protein that can be used in the treatment of a subject where the expression of a peptide or protein would be of benefit, e.g., in ameliorating the symptoms of a disease or disorder. For example, a pharmaceutically active protein can replace or augment protein expression in a cell which does not normally express a protein or which misexpresses a protein, e.g., a pharmaceutically active protein can compensate for a mutation by supplying a desirable protein. In addition, a “pharmaceutically active peptide or protein” can produce a beneficial outcome in a subject, e.g., can be used to produce a protein to which vaccinates a subject against an infectious disease. Preferably, a “pharmaceutically active peptide or protein” has a positive or advantageous effect on the condition or disease state of a subject when administered to the subject in a therapeutically effective amount. Preferably, a pharmaceutically active peptide or protein has curative or palliative properties and may be administered to ameliorate, relieve, alleviate, reverse, delay onset of or lessen the severity of one or more symptoms of a disease or disorder. A pharmaceutically active peptide or protein may have prophylactic properties and may be used to delay the onset of a disease or to lessen the severity of such disease or pathological condition. The term “pharmaceutically active peptide or protein” includes entire proteins or polypeptides, and can also refer to pharmaceutically active fragments thereof. It can also include pharmaceutically active analogs of a peptide or protein. The term “pharmaceutically active peptide or protein” includes peptides and proteins that are antigens, i.e., the peptide or protein elicits an immune response in a subject which may be therapeutic or partially or fully protective.

Examples of pharmaceutically active proteins include, but are not limited to, cytokines and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, seletins, homing receptors, T cell receptors, immunoglobulins, soluble major histocompatibility complex antigens, immunologically active antigens such as bacterial, parasitic, or viral antigens, allergens, autoantigens antibodies), hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthetic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, von Wilebrand factor, antithrombin III, glucocerebrosidase, erythropoietin granulocyte colony stimulating factor (GCSF) or modified Factor VIII, anticoagulants and the like.

In one embodiment, the pharmaceutically active protein according to the invention is a cytokine which is involved in regulating lymphoid homeostasis, preferably a cytokine which is involved in and preferably induces or enhances development, priming, expansion, differentiation and/or survival of T cells. In one embodiment, the cytokine is an interleukin. In one embodiment, the pharmaceutically active protein according to the invention is an interleukin selected from the group consisting of IL-2, IL-7, IL-12, IL-15, and IL-21.

The term “immunologically active compound” relates to any compound altering an immune response, preferably by inducing and/or suppressing maturation of immune cells, inducing and/or suppressing cytokine biosynthesis, and/or altering humoral immunity by stimulating antibody production by B cells. Immunologically active compounds possess potent immunostimulating activity including, but not limited to, antiviral and antitumor activity, and can also down-regulate other aspects of the immune response, for example shifting the immune response away from a TH2 immune response, which is useful for treating a wide range of TH2 mediated diseases. Immunologically active compounds can be useful as vaccine adjuvants.

In one embodiment, RNA that codes for an antigen such a disease-associated antigen is administered to a mammal, in particular if treating a mammal having a disease involving the antigen is desired. The RNA is preferably taken up into the mammal's antigen-presenting cells (monocytes, macrophages. dendritic cells or other cells). An antigenic translation product of the RNA is formed and the product is displayed on the surface of the cells for recognition by T cells. In one embodiment, the antigen or a product produced by optional procession thereof is displayed on the cell surface in the context of MHC molecules for recognition by T cells through their T cell receptor leading to their activation.

In the context of the present invention the terms “salt(s)” and “electrolyte(s)” are used interchangeably and mean a compound that at least partially dissociates into its respective counter ions in water.

According to the present invention, the term “mM electrolytes” means the concentration in 10⁻³ mol per liter of the sum of all electrolytes (including inorganic salts such as NaCl, KCl, NaH₂PO₄, Na₂HPO₄, KH₂PO₄, K₂HPO₄, MgCl₂, MnCl₂, Na₂SO₄, K₂SO₄, MgSO₄ and salts such Tris-HCl, EDTA, Hepes, etc.) in the solutions used to resuspend or to dilute the RNA stock solutions and in the solutions used to dilute Protamine stock solutions.

It should be noted that, once the particles of the present invention are formed, the specific salt (or electrolyte) concentration conditions used for preparing the particles need not to be further maintained. Thus, the particles can be further processed, e.g. eventually recovered by centrifugation and diluted, dissolved or dispersed in a medium, preferably a pharmaceutically acceptable excipient, vehicle and/or diluent, in particular in an isotonic medium such as saline, Ringer or Ringer Lactate solution.

Interferons are important cytokines characterized by antiviral, antiproliferative and immunomodulatory activities. Interferons are proteins that alter and regulate the transcription of genes within a cell by binding to interferon receptors on the regulated cell's surface, thereby preventing viral replication within the cells. The interferons can be grouped into two types. IFN-gamma is the sole type II interferon; all others are type I interferons. Type I and type II interferons differ in gene structure (type II interferon genes have three exons; type I, chromosome location (in humans, type II is located on chromosome-12; the type I interferon genes are linked and on chromosome-9), and the types of tissues where they are produced (type I interferons are synthesized ubiquitously, type II by lymphocytes). Type I interferons competitively inhibit each others binding to cellular receptors, while type II interferon has a distinct receptor. According to the invention, the term “interferon” or “IFN” preferably relates to type I interferons, in particular IFN-alpha and IFN-beta.

The present invention is useful to prime, activate or strengthen the immunity in certain disease states, in particular in the case of chronic diseases, such as cancer or infectious diseases, in particular persistent virus infections. Thus, the method of the present invention is useful in the treatment of said disease states. The method of the present invention is particularly suitable for inducing production, or increasing the level of interferons, in particular interferon-alpha and/or interferon-beta. Thus, the method of the present invention may be used to supplement interferon-alpha treatment and/or interferon-beta treatment, or to increase interferon-alpha and/or interferon-beta in a subject.

According to the invention, the term “disease” refers to any pathological state, including cancer diseases. Cancer (medical term: malignant neoplasm) is a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor, i.e. a swelling or lesion formed by an abnormal growth of cells (called neoplastic cells or tumor cells), but some, like leukemia, do not. The term “cancer” according to the invention comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. Examples thereof are lung carcinomas, mamma carcinomas, prostate carcinomas, colon carcinomas, renal cell carcinomas, cervical carcinomas, or metastases of the cancer types or tumors described above. The term cancer according to the invention also comprises cancer metastases.

Examples of cancers treatable with the present invention include malignant melanoma, all types of carcinoma (colon, renal cell, bladder, prostate, non-small cell and small cell lung carcinoma, etc.), lymphomas, sarcomas, blastomas, gliomas, myeiomas, etc.

Malignant melanoma is a serious type of skin cancer. It is due to uncontrolled growth of pigment cells, called melanocytes.

According to the invention, a “carcinoma” is a malignant tumor derived from epithelial cells. This group represents the most common cancers, including the common forms of breast, prostate, lung and colon cancer.

Lymphoma and leukemia are malignancies derived from hematopoietic (blood-forming) cells.

A sarcoma is a cancer that arises from transformed cells in one of a number of tissues that develop from embryonic mesoderm. Thus, sarcomas include tumors of bone, cartilage, fat, muscle, vascular, and hematopoietic tissues.

Blastic tumor or blastoma is a tumor (usually malignant) which resembles an immature or embryonic tissue. Many of these tumors are most common in children.

A glioma is a type of tumor that starts in the brain or spine. It is called a glioma because it arises from glial cells. The most common site of gliomas is the brain.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor and the regional lymph node system.

Examples of infectious diseases treatable with the present invention include viral infectious diseases, such as AIDS (HIV), hepatitis A, B or C, herpes, herpes zoster (chicken-pox), German measles (rubella virus), yellow fever, dengue etc. flaviviruses, influenza viruses, hemorrhagic infectious diseases (Marburg or Ebola viruses), bacterial infectious diseases, such as Legionnaire's disease (Legionella), gastric ulcer (Helicobacter), cholera (Vibrio), infections by E. coli, Staphylococci, Salmonella or Streptococci (tetanus); infections by protozoan pathogens such as malaria, sleeping sickness, leishmaniasis; toxoplasmosis, i.e. infections by Plasmodium, Trypanosoma, Leishmania and Toxoplasma; or fungal infections, which are caused e.g. by Cryptococcus neoformans, Histoplasma capsulatum, Coccidioides immitis, Blastomyces dermatitidis or Candida albicans).

The present invention is also useful in treating allergies.

The method of the present invention can also be used in conjunction with other therapeutic agents which can be administered prior to, simultaneously with or after administration of the compositions used according to the present invention. Such therapeutic agents include chemotherapeutic drugs for cancer patients, e.g. gemcitabine, etopophos, cis-platin, carbo-platin, antiviral agents, anti-parasite agents or anti-bacterial agents.

In particular, the present invention can also be used in conjunction with an immunotherapeutic agent, preferably an immunotherapeutic agent inducing or effecting a targeted, i.e. specific, immune reaction. Such immunotherapeutic agents include agents directed against a disease-associated antigen such as therapeutic antibodies or agents inducing an immune response directed against a disease-associated antigen or cells expressing a disease-associated antigen. Useful immunotherapeutic agents include proteins or peptides inducing a B cell or T cell response against the disease-associated antigen or cells expressing the disease-associated antigen. These proteins or peptides may comprise a sequence essentially corresponding to or being identical to the sequence of the disease-associated antigen or one or more fragments thereof. In one embodiment, the protein or peptide comprises the sequence of an MHC presented peptide derived from the disease-associated antigen. Instead of administering the protein or peptide it is also possible to administer nucleic acids, preferably mRNA, encoding the protein or peptide. Accordingly, the present invention may be used in genetic vaccination, wherein an immune response is stimulated by introduction into a subject a suitable nucleic acid molecule (DNA or mRNA) which codes for an antigen or a fragment thereof. This mRNA may be present within immunostimulating particles described herein and may be immunostimulating RNA or other RNA.

In one embodiment, a disease-associated antigen is a tumor-associated antigen. In this embodiment, the present invention may be useful in treating cancer or cancer metastasis. Preferably, the diseased organ or tissue is characterized by diseased cells such as cancer cells expressing a disease-associated antigen and/or being characterized by association of a disease-associated antigen with their surface. Immunisation with intact or substantially intact tumor-associated antigen or fragments thereof such as MHC class I and class II peptides or nucleic acids, in particular mRNA, encoding such antigen or fragment makes it possible to elicit a MHC class I and/or a class II type response and thus, stimulate T cells such as CD8+ cytotoxic T lymphocytes which are capable of lysing cancer cells and/or CD4+ T cells. Such immunization may also elicit a humoral immune response (B cell response) resulting in the production of antibodies against the tumor-associated antigen. Furthermore, antigen presenting cells (APC) such as dendritic cells (DCs) can be loaded with MHC class I—presented peptides directly or by transfection with nucleic acids encoding tumor antigens or tumor antigen peptides in vitro and administered to a patient.

According to the present invention, a tumor-associated antigen preferably comprises any antigen which is characteristic for tumors or cancers as well as for tumor or cancer cells with respect to type and/or expression level. In one embodiment, the term “tumor-associated antigen” relates to proteins that are under normal conditions, i.e. in a healthy subject, specifically expressed in a limited number of organs and/or tissues or in specific developmental stages, for example, the tumor-associated antigen may be under normal conditions specifically expressed in stomach tissue, preferably in the gastric mucosa, in reproductive organs, e.g., in testis, in trophoblastic tissue, e.g., in placenta, or in germ line cells, and are expressed or aberrantly expressed in one or more tumor or cancer tissues. In this context, “a limited number” preferably means not more than 3, more preferably not more than 2 or 1. The tumor-associated antigens in the context of the present invention include, for example, differentiation antigens, preferably cell type specific differentiation antigens, i.e., proteins that are under normal conditions specifically expressed in a certain cell type at a certain differentiation stage, cancer/testis antigens, i.e., proteins that are under normal conditions specifically expressed in testis and sometimes in placenta, and germ line specific antigens. In the context of the present invention, the tumor-associated antigen is preferably associated with the cell surface of a cancer cell and is preferably not or only rarely expressed in normal tissues. Preferably, the tumor-associated antigen or the aberrant expression of the tumor-associated antigen identifies cancer cells. In the context of the present invention, the tumor-associated antigen that is expressed by a cancer cell in a subject, e.g., a patient suffering from a cancer disease, is preferably a self-protein in said subject. In preferred embodiments, the tumor-associated antigen in the context of the present invention is expressed under normal conditions specifically in a tissue or organ that is non-essential, i.e., tissues or organs which when damaged by the immune system do not lead to death of the subject, or in organs or structures of the body which are not or only hardly accessible by the immune system. The amino acid sequence of the tumor-associated antigen may be identical between the tumor-associated antigen which is expressed in normal tissues and the tumor-associated antigen which is expressed in cancer tissues or mutations may be found in the tumor tissue. Preferably, a tumor-associated antigen is presented in the context of MHC molecules by a cancer cell in which it is expressed.

Examples for differentiation antigens which ideally fulfill the criteria for tumor-associated antigens as contemplated by the present invention as target structures in tumor immunotherapy, in particular, in tumor vaccination are the cell surface proteins of the ciaudin family, such as CLDN6 and CLDN18.2. These differentiation antigens are expressed in tumors of various origins, and are particularly suited as target structures in connection with antibody-mediated cancer immunotherapy due to their selective expression (no expression in a toxicity relevant normal tissue) and localization to the plasma membrane.

Further examples for antigens that may be useful in the present invention are wild type or mutated p53, ART-4, BAGE, beta-catenin/m, Bcr-abL CAMEL, CAP-1, CASP-8, CDC27/m, CDK4/m, CEA, CLAUDIN-12, c-MYC, CT, Cyp-B, DAM, ELF2M, ETV6-AML1, G250, GAGE, GnT-V, Gap100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2, -3, NA88-A, NF1, NY-ESO-1, NY-BR-1, p190 minor BCR-abL, Pm1/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT, preferably WT-1.

The compositions used according to the present invention may take the form of a vaccine comprising the danger signal such as Protamine-RNA particles and at least one antigen such as an antigen as discussed above or an immunogenic fragment thereof, or a nucleic acid, in particular mRNA, encoding said antigen or fragment.

An “antigen” is to be understood as meaning any structure which can cause the formation of antibodies and/or the activation of a cellular immune response. Examples of antigens are polypeptides, proteins, cells, cell extracts, carbohydrates/polysaccharides, polysaccharide conjugates, lipids, and glycolipids. These antigens may be tumor antigens or viral, bacterial, fungal and protozoological antigens or allergens. The term “antigen” also includes derivatized antigens as secondary substance which becomes antigenic—and sensitizing—only through transformation (e.g., intermediately in the molecule, by completion with body protein), and conjugated antigens which, through artificial incorporation of atomic groups (e.g., isocyanates, diazonium salts), display a new constitutive specificity. The antigen may be administered in the form of a hapten coupled to a suitable carrier. Suitable carriers are known to those ordinarily skilled in the art and include e.g. human serum albumin (HSA), polyethylene glycols (PEG). The hapten may be coupled to the carrier by processes well-known in the prior art, e.g. in the case of a polypeptide carrier via an amide bond to a Lys residue. The antigen may be also coated onto the Protamine-RNA particles.

By “treat” is meant to administer a compound or composition as described herein to a subject in order to prevent or eliminate a disease, including reducing the size of a tumor or the number of tumors in a subject; arrest or slow a disease in a subject; inhibit or slow the development of a new disease in a subject; decrease the frequency or severity of symptoms and/or recurrences in a subject who currently has or who previously has had a disease; and/or prolong, i.e. increase the lifespan of the subject.

In particular, the term “treatment of a disease” includes curing, shortening the duration, ameliorating, preventing, slowing down or inhibiting progression or worsening, or preventing or delaying the onset of a disease or the symptoms thereof.

The term “immunotherapy” relates to a treatment preferably involving a specific immune reaction and/or immune effector function(s).

The term “immunization” or “vaccination” describes the process of treating a subject for therapeutic or prophylactic reasons.

The term “subject” relates to mammals. For example, mammals in the context of the present invention are humans, non-human primates, domesticated animals such as dogs, cats, sheep, cattle, goats, pigs, horses etc., laboratory animals such as mice, rats, rabbits, guinea pigs, etc. as well as animals in captivity such as animals of zoos. The term “subject” as used herein also includes humans.

The compositions described herein are preferably sterile and contain an effective amount of the active components, in particular danger signal such as particles described herein, and optionally of further agents as discussed herein such as therapeutic agents and antigens to generate the desired reaction or the desired effect.

The compositions described herein may be formulated as an emulsion containing an oil such as Montanide®.

The compositions described herein may also comprise an additional immunomodulating agent such as anti-CTL-A4 or anti-PD1 or anti-PDL1 or anti-regulatory T-cell reagents such as an anti-CD25 antibody or cyclophosphamide. The compositions described herein may be administered together with supplementing immunity-enhancing substances such as one or more adjuvants and may comprise one or more immunity-enhancing substances to further increase their effectiveness, preferably to achieve a synergistic effect of immunostimulation.

The term “adjuvant” relates to compounds which prolong or enhance or accelerate an immune response. Various mechanisms are possible in this respect, depending on the various types of adjuvants. For example, compounds which allow the maturation of the DC, e.g. lipopolysaccharides or CD40 ligand, form a first class of suitable adjuvants. Generally, any agent which influences the immune system of the type of a “danger signal” (LPS, GP96, dsRNA etc.) or cytokines, such as GM-CSF, can be used as an adjuvant which enables an immune response to be intensified and/or influenced in a controlled manner. CpG oligodeoxynucleotides, double stranded RNA and imiquimod/resiquimod can also be used in this context. Particularly preferred adjuvants are cytokines, such as monokines, lymphokines, interleukines or chemokines, e.g. IL-1, IL-2, IL-3, IL-4, IL-5, IL-6, IL-7, IL-8, IL-9, IL-10, IL-12, INFα, INF-γ, GM-CSF, LT-α, or growth factors, e.g. hGH. Further known adjuvants are aluminium hydroxide, Freund's adjuvant or oil such as Montanide®, most preferred

Montanide® ISA51. Lipopeptides, such as Pam3Cys, are also suitable for use in the present invention.

Compositions described herein are usually provided in a uniform dosage form and may be prepared in a manner known per se. The compositions may e.g. be in the form of a solution or suspension.

The compositions described herein may comprise salts, buffer substances, preservatives, carriers, diluents and/or excipients all of which are preferably pharmaceutically acceptable. The term “pharmaceutically acceptable” refers to the non-toxicity of a material which does not interact with the action of the active component of the pharmaceutical composition.

Salts which are not pharmaceutically acceptable may used for preparing pharmaceutically acceptable salts and are included in the invention. Pharmaceutically acceptable salts of this kind comprise in a non limiting way those prepared from the following acids: hydrochloric, hydrobromic, sulfuric, nitric, phosphoric, maleic, acetic, salicylic, citric, formic, malonic, succinic acids, and the like. Pharmaceutically acceptable salts may also be prepared as alkali metal salts or alkaline earth metal salts, such ^{as} sodium salts, potassium salts or calcium salts.

Suitable buffer substances for use in the invention include acetic acid in a salt, citric acid in a salt, boric acid in a salt and phosphoric acid in a salt.

Suitable preservatives for use in the invention include benzalkonium chloride, chlorobutanol, paraben and thimerosal.

An injectible formulation may comprise a pharmaceutically acceptable excipient such as Ringer Lactate.

The term “carrier” refers to an organic or inorganic component, of a natural or synthetic nature, in which the active component is combined in order to facilitate, enhance or enable application. According to the invention, the term “carrier” also includes one or more compatible solid or liquid fillers, diluents or encapsulating substances, which are suitable for administration to a patient.

Possible carrier substances for parenteral administration are e.g. sterile water, Ringer, Ringer lactate, sterile sodium chloride solution, polyalkylene glycols, hydrogenated naphthalenes and, in particular, biocompatible lactide polymers, lactide/glycolide copolymers or polyoxyethylene/polyoxy- propylene copolymers.

The term “excipient” when used herein is intended to indicate all substances which may be present in a composition described herein and which are not active ingredients such as, e.g., carriers, binders, lubricants, thickeners, surface active agents, preservatives, emulsifiers, buffers, flavoring agents, or colorants.

The agents and compositions described herein may be administered via any conventional route, such as by parenteral administration including by injection or infusion. Administration is preferably parenterally, e.g. intravenously, intraarterially, subcutaneously, intradermally or intramuscularly.

Compositions suitable for parenteral administration usually comprise a sterile aqueous or nonaqueous preparation of the active compound, which is preferably isotonic to the blood of the recipient. Examples of compatible carriers and solvents are Ringer solution and isotonic sodium chloride solution. In addition, usually sterile, fixed oils are used as solution or suspension medium.

The agents and compositions described herein are administered in effective amounts. An “effective amount” refers to the amount which achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition.

An effective amount of an agent or composition described herein will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses administered of the agents described herein may depend on various of such parameters. In the case that a reaction in a patient is insufficient with an initial dose, higher doses (or effectively higher doses achieved by a different, more localized route of administration) may be used.

The following examples are intended to illustrate preferred embodiments of the invention and should not be interpreted to limit the scope of the invention as defined in the claims.

### EXAMPLES

### Example 1

### Synergistic Production of Type I Interferon but not of TNF-alpha by a Double Injection of Protamine-RNA Particles

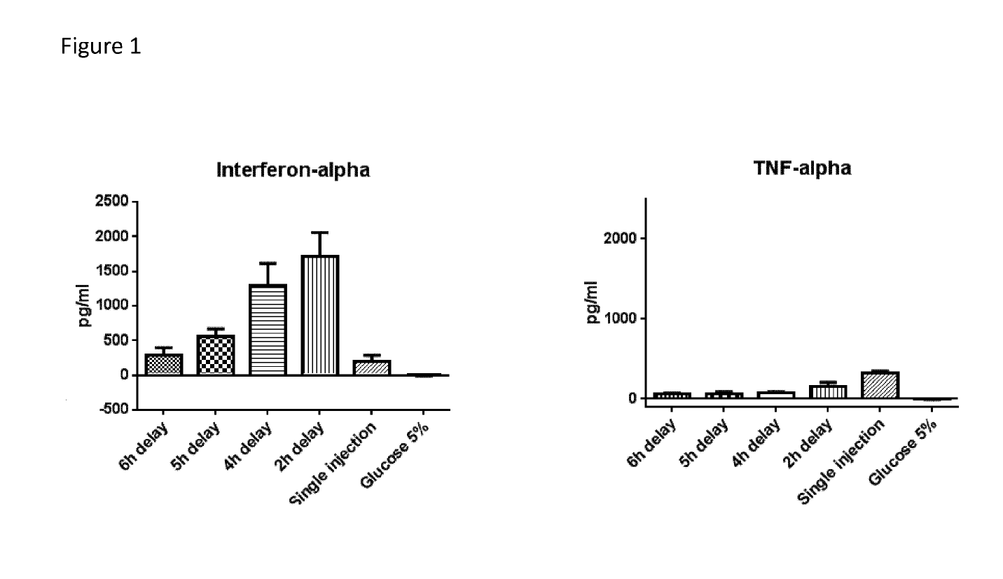

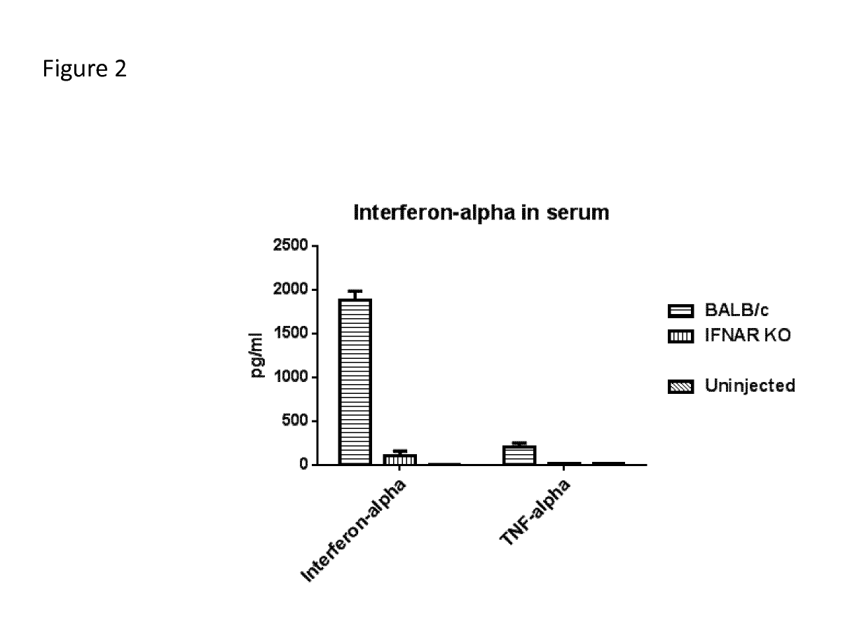

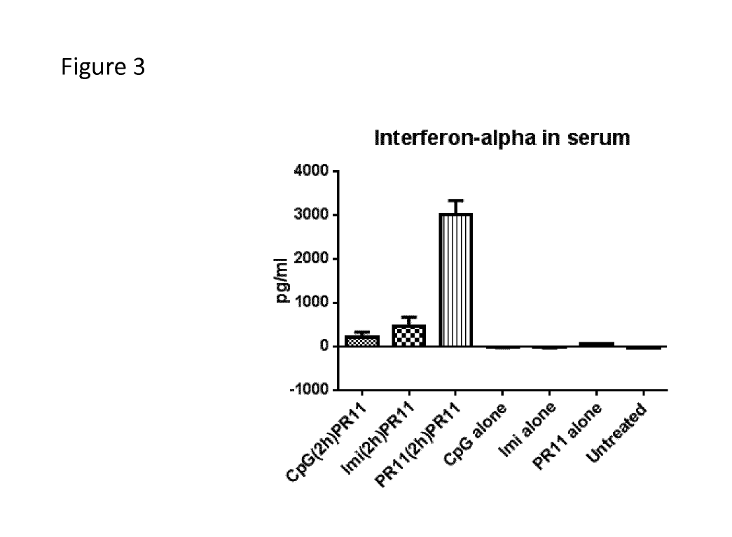

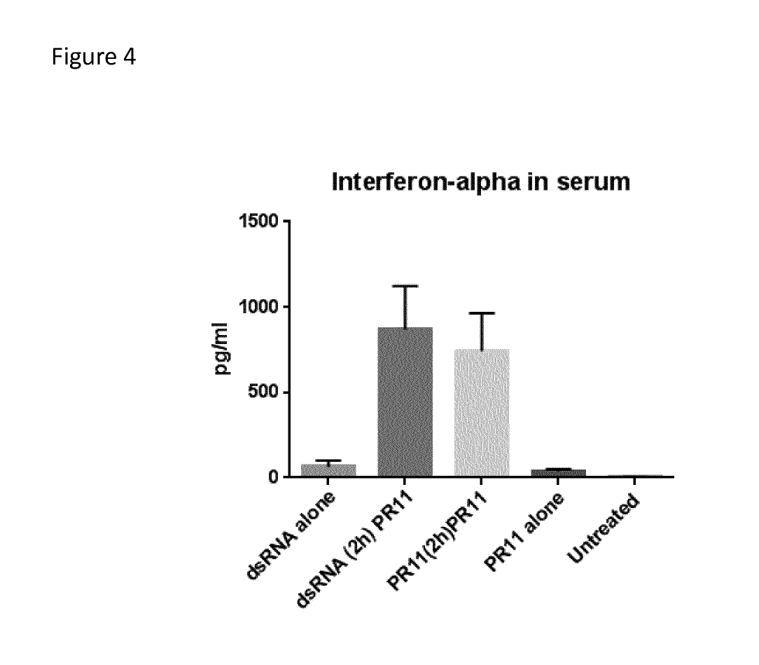

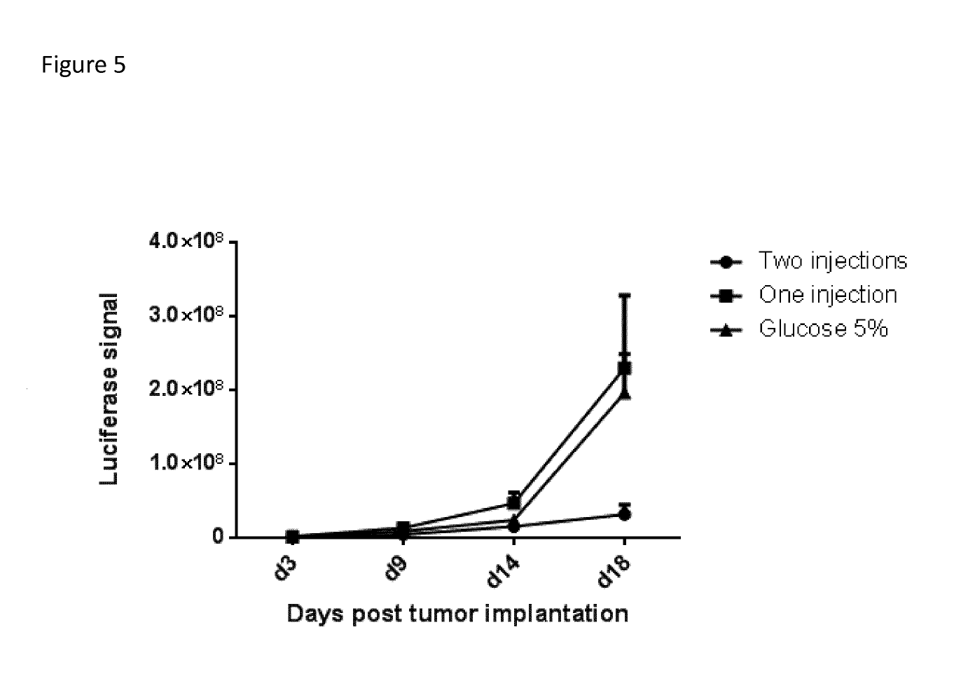

An RNA is synthesized and purified. The product is then lyophilized and resuspended at 0.5 mg/ml in pure water. Protamine !PEX 5000 is diluted 28 times in pure water to provide a solution of Protamine at approximately 0.5 mg/ml in low salt. One volume of RNA is mixed with one volume of Protamine. immediate and intensive mixing is performed for example by pipeting up and down or by vortexing. The formulation is left for ten minutes at room temperature and is then further diluted with an adequate amount of 40% Glucose to reach a final concentration of 5% glucose. The solution is further diluted with 5% Glucose in order to achieve a concentration of RNA (and of Protamine) of 10 micrograms in 70 microliters. Mice get one injection intravenous of 140 microliters of the Protamine-RNA solution or two intra-venous injections of 70 microliters of the solution, injections being done two or four or five or six hours apart. In those conditions, as shown in FIG. 1, a synergistic production of interferon-alpha, but not of TNF-alpha, is detectable in serum collected three hours after the (last) injection when the two injections are done 2 or 4 hours apart (up to 5 hours apart).

### Example 2

### Dependency on the Interferon-Alpha Receptor 1

Using the same formulation of Protamine-RNA as in example 1 and a double injection schedule where the intra-venous administrations of 70 microliters are separated by two hours and serum collected three hours after the second injection, it appears, as depicted in FIG. 2, that synergistic production of interferon-alpha depends on IFNAR (it is not seen in mice deficient for IFNAR-1). This suggests that inducers (even weak) of type I interferon in a functional organism (having functional type I interferon receptors) as well as purified or recombinant type I interferon could sensitize the organism to respond strongly (in term of interferon production) to a second injection of Protamine-RNA particles.

### Example 3

### Danger Signals can Sensitize to the Subsequent Injection of Protamine-RNA Particles

70 microliters of Protamine-RNA particles as described in example 1 induce strong (synergistic) interferon production when they are administered 2 hours after a first injection of the same amount of Protamine-RNA particles or after injection of other danger signals such as unmethylated DNA (“CpG ODN”) or imiquimod (both in FIG. 3) or dsRNA (FIG. 4). Thus, Protamine-RNA particles can be injected after (or before) another danger signal in order to get a synergistic production of type I interferon.

### Example 4

### Cure of Established Tumors using a Double Injection Schedule of Protamine-RNA Particles.

As depicted in FIG. 5, mice with established lung metastasis were showing a delay in tumor development when they were treated using two cycles (one week apart) of a “synergistic” injection protocol (two, 2 hours apart, intra-venous injections of 70 microlitres of Protamine-RNA particles, as prepared in example 1) but not when treated using two cycles (one week apart) of a “bulk” protocol (one intra-venous injection of 140 microlitres of Protamine-RNA particles as described in example 1). Thus, the schedule of a double injection within a few hours (less than 6) is necessary to trigger an efficacious anti-cancer immunity by Protamine-RNA particles.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. Furthermore, the teachings and disclosures of all references cited herein are expressly incorporated in their entireties by reference.

1. A method for inducing an immune response in a subject comprising administering to said subject a second composition after administration of a first composition, the first composition and the second composition each comprising (i) a danger signal; and (ii) a pharmaceutically acceptable carrier,
wherein the first composition and the second composition are administered within a time period such that a synergistic immune response is induced.

2. The method of claim 1, wherein the time period is about 6 hours.

3. The method of claim 1, wherein the danger signal is a Toll-like receptor agonist and/or wherein the danger signal induces type I interferon.

4. The method of claim 3, wherein the Toll-like receptor agonist is selected from the group consisting of a particle comprising RNA, in which the RNA is associated with a cationic polymer or lipid or both a cationic polymer and lipid; double-stranded RNA;
unmethylated DNA containing CpG motifs; imiquimod; and resiquimod.

5. The method of claim 4, wherein the cationic polymer is selected from the group consisting of Protamine, polyethyleneimine, poly-L-lysine, poly-L-arginine and histone.

6. The method of claim 5, wherein the particle comprises RNA and Protamine.

7. The method of claim 6, wherein the Protamine-RNA particle is a Protamine-RNA nanoparticle having a size in the range from about 10 nm to about 990 nm.

8. The method of claim 7, wherein the Protamine-RNA nanoparticle has a polycation:RNA mass ratio in the range from about 16:1 to about 1:2.

9. The method of claim 1, wherein the danger signal in the first and/or second composition is a Protamine-RNA nanoparticle.

10. The method of claim 4, wherein the RNA comprised in the particle is an oligonucleotide or a messenger RNA.

11. The method of claim 4, wherein the RNA comprised in the particle comprises at least one U nucleotide or at least one G nucleotide, or at least one U nucleotide and at least one G nucleotide.

12. The method of claim 4, wherein the RNA comprised in the particle is modified RNA.

13. The method of claim 1, wherein the time period is about 5 hours.

14. The method of claim 1, wherein the induced immune response is detected by an at least 2-fold increase in type I interferon expression in serum obtained from the subject after administration of both the first composition and the second composition as compared to administration of only one of the compositions.

15. The method of claim 1, wherien the induced immune response shows no increase or a decrease in TNF-alpha expression in serum obtained from the subject after administration of both the first composition and the second composition as compared to administration of only one of the compositions.

16. The method of claim 1, wherien the first composition and/or the second composition further comprises an antigen.

17. A kit comprising a first container and a second container, wherein
the first container contains a first composition comprising (i) a danger signal; and (ii) a pharmaceutically acceptable carrier; and
the second container contains a second composition comprising (i) a danger signal; and (ii) a pharmaceutically acceptable carrier.

18.-19. (canceled)

20. The method of claim 1, wherein the subject has cancer, and the method treats the cancer.
